(19) United States
(12) Patent Application Publication
(10) Pub. No.: US 2026/0325521 A1
(43) Pub. Date: Oct. 22, 2026

(54) TREATMENT OF IDIOPATHIC PULMONARY FIBROSIS WITH ENSIFENTRINE

(71) Applicant: Verona Pharma Limited, London (GB)

(72) Inventors: Tara Renae Rheault, Cary, NC (US); Kathleen Rickard, Durham, NC (US)

(73) Assignee: Verona Pharma Limited, London (GB)

(21) Appl. No.: 19/122,559

(22) PCT Filed: Oct. 19, 2023
(86) PCT No.: PCT/GB2023/052709

§ 371 Date: Apr. 18, 2025

Related U.S. Application Data
(60) Provisional application No. 63/417,762, filed on Oct. 20, 2022.
(60) Provisional application No. 63/580,726, filed on Sep. 6, 2023.

(51) Int. Cl. A61K 31/519; A61K 9/00; A61K 31/4418; A61K 31/496; A61P 11/00

(52) U.S. Cl. CPC A61K 31/519; A61K 9/0073; A61K 31/4418; A61K 31/496; A61P 11/00

(57) ABSTRACT

The present invention relates to a compound for use in a method of treating idiopathic pulmonary fibrosis in a patient, which compound is ensifentrine or a pharmaceutically acceptable salt thereof, wherein the method comprises administering the compound to the patient by inhalation. The present invention relates to a compound for use in a method of treating pulmonary fibrosis in a patient by improving lung function, or by preventing or reducing a reduction in lung function, which compound is ensifentrine or a pharmaceutically acceptable salt thereof, wherein the method comprises administering the compound to the patient by inhalation.

### FIELD OF THE INVENTION

The present invention relates to the treatment of pulmonary fibrosis (PF), and in particular the treatment of idiopathic pulmonary fibrosis (IPF).

### BACKGROUND OF THE INVENTION

Ensifentrine (N-(2-{(2E)-9,10-dimethoxy-4-oxo-2-[(2,4,6-trimethylphenyl)imino]-6,7-dihydro-2H-pyrimido[6,1-a]isoquinolin-3(4H)-yl}ethyl)urea, also known as RPL554) is a dual PDE3/PDE4 inhibitor (including inhibition of PDE4B) and is described in WO 00/58308 A1.

As a combined phosphodiesterase 3/4 (PDE3/PDE4) inhibitor, ensifentrine has both bronchodilatory and anti-inflammatory activity and is useful in the treatment of respiratory disorders including chronic obstructive pulmonary disease (COPD). The chemical structure of ensifentrine is shown below.

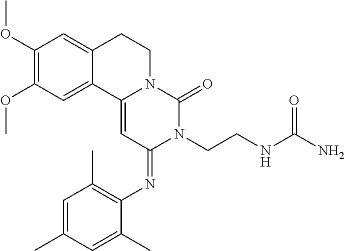 [SMILES: COc1cc2c(cc1OC)-c1c/c(=N\c3c(C)cc(C)cc3C)n(CCNC(N)=O)c(=O)n1CC2]

Pulmonary fibrosis is a disease associated with scarring of the tissue of the lungs. The scarring causes hardening of the lung tissue, which leads to symptoms such as shortness of breath. Pulmonary fibrosis can be caused by a number of factors, including bacterial or viral infection and cigarette smoke. Most pulmonary fibrosis is idiopathic, however, and has no known cause. Idiopathic pulmonary fibrosis (IPF) is a progressive and debilitating disease which affects around five million people globally.

There is no cure for pulmonary fibrosis and limited treatment options. Most therapies focus on slowing the progression of the disease. Known treatments include the anti-fibrotic agents Esbriet (pirfenidone) and Ofev (nintedanib), both of which are administered orally. However, oral treatments for IPF are associated with non-trivial side-effects (including diarrhoea), making tolerability an issue.

There is a need to develop further treatments for pulmonary fibrosis, including for IPF. In particular, it is desirable to develop treatments with reduced side effects and/or better patient tolerability.

### SUMMARY OF THE INVENTION

It is a surprising finding of the present invention that ensifentrine may be used to treat pulmonary fibrosis, and in particular idiopathic pulmonary fibrosis, by inhaled administration. Inhaled ensifentrine has also been found to avoid side effects such as diarrhoea.

The invention accordingly provides a compound for use in a method of treating idiopathic pulmonary fibrosis in a patient, which compound is ensifentrine or a pharmaceutically acceptable salt thereof, wherein the method comprises administering the compound to the patient by inhalation.

Also provided by the invention is a compound for use in a method of treating pulmonary fibrosis in a patient by improving lung function, or by preventing or reducing a reduction in lung function, which compound is ensifentrine or a pharmaceutically acceptable salt thereof, wherein the method comprises administering the compound to the patient by inhalation.

The invention further provides a compound for use in a method of treating pulmonary fibrosis in a patient by preventing or reducing fibrosis, which compound is ensifentrine or a pharmaceutically acceptable salt thereof, wherein the method comprises administering the compound to the patient by inhalation.

The invention further provides a composition comprising (i) a compound which is ensifentrine or a pharmaceutically acceptable salt and (ii) an anti-fibrotic agent.

### BRIEF DESCRIPTION OF THE FIGURES

FIG. 1 shows the effect of inhaled ensifentrine on blood IL-6 and IL-8 inflammatory biomarkers.

### DETAILED DESCRIPTION OF THE INVENTION

The compound may improve lung function in a patient with pulmonary fibrosis. For instance, the compound may increase forced vital capacity (FVC) in a patient with pulmonary fibrosis. For instance, the FVC of the patient one month after the compound is first administered may be at least 0.2%, at least 0.5%, at least 1% or at least 2% greater than the FVC of the patient on the date the compound is first administered. FVC may be measured by standard spirometry techniques. Typically, as used herein, FVC is determined as set out in the article Standardisation of Spirometry, Eur Resp J 2005; 26; 319-338.

The compound may prevent a reduction in lung function in a patient with pulmonary fibrosis. For instance, the compound may prevent (or reduce) a reduction in forced vital capacity (FVC) in a patient with pulmonary fibrosis. As such, the FVC of the patient one month after the compound is first administered may be no more than 0.2%, no more than 0.5%, no more than 1% or no more than 2% less than the FVC of the patient on the date the compound is first administered.

The compound may act as an anti-fibrotic. As such, the compound may prevent or reduce fibrosis, and in particular the compound may prevent or reduce fibrosis of lung tissue. The compound may therefore prevent formation of fibrosis or scarring of lung tissue in a patient or the compound may reduce the amount of fibrosis or scar tissue in the lungs of the patient. The compound may improve lung function by preventing or reducing fibrosis of lung tissue.

The compound may be for use in a method of treating pulmonary fibrosis in a patient by preventing or reducing fibrosis. For instance, the method may comprise treating pulmonary fibrosis in a patient by reducing fibrosis in the lungs of the patient.

The compound may cause a reduction in TGF-β concentration in the patient, for instance a reduction in lung TGF-β concentration in the patient.

The pulmonary fibrosis may be caused by infection or an external chemical irritant. The pulmonary fibrosis may be fibrosing alveolitis, for instance cryptogenic fibrosing alveolitis. The pulmonary fibrosis is preferably idiopathic pulmonary fibrosis.

Fibrosis of the lungs associated with pulmonary fibrosis in a patient may be measured by determining a decline in lung function of the patient. The decline in lung function may be a decline in forced vital capacity (FVC) of the patient. The compound may prevent a decline in FVC of the patient by preventing fibrosis of lung tissue. The compound may improve FVC of the patient by reducing fibrosis of lung tissue.

The FVC of the patient may increase following administration of the compound. For instance, the FVC of the patient may be increased by at least 10 mL or at least 20 mL one month after the first inhaled administration of the compound (compared with FVC at the first administration of the compound). The FVC of the patient may decrease by no more than 30 mL or no more than 50 mL one month after the first inhaled administration of the compound (compared with FVC at the first administration of the compound).

The compound may decrease the frequency and/or severity of pulmonary fibrosis (PF) exacerbations in the patient. The compound may decrease the frequency of PF exacerbations. For instance, the patient may suffer two or fewer (for instance one or zero) PF exacerbations per year while being treated with the compound, for instance as a maintenance therapy. The number of PF exacerbations experienced by the patient per year during treatment with the compound may be one to three fewer than the number of PF exacerbations experienced by the patient per year prior to treatment with the compound.

The compound may increase the time to a first PF exacerbation in the patient. Accordingly, the patient may not have yet experienced a COPD exacerbation and the compound may increase the time until the patient experiences a first PF exacerbation (i.e. the first PF exacerbation is delayed). The compound may accordingly reduce the risk of PF exacerbations in a patient suffering from pulmonary fibrosis.

In some cases, the patient has suffered one or more PF exacerbations in the year preceding first administration of the compound. For instance, the patient may have suffered two or more PF exacerbations in the year preceding first administration of the compound. The patient may for instance have suffered from at least one severe PF exacerbation (i.e. requiring hospital treatment) in the preceding year. The patient may have suffered one or more PF exacerbations in the six months preceding first administration of the compound, or one or more PF exacerbations in the one month preceding first administration of the compound.

The PF exacerbation may be an acute PF exacerbation. For instance, the PF exacerbation may be an acute exacerbation of IPF (AE-IPF). AE-IPF is defined as a sudden acceleration of the disease or an idiopathic acute injury superimposed on the diseased lung that leads to a significant decline in lung function.

The patient may be suffering from idiopathic pulmonary fibrosis (IPF) and the compound may decrease the frequency and/or severity of acute exacerbation of IPF (AE-IPF) in the patient.

The patient may be male. The patient may be female. The patient may have an age of greater than or equal to 65 years. The patient may have an age of less than 65 years. The patient may be taking a background medication selected from one or more of an anti-fibrotic agent, a long-acting muscarinic antagonist (LAMA), a long-acting beta-agonist (LABA) and an inhaled corticosteroid (ICS). In some cases, the patient is not receiving a background medication. For instance, the patient may not be taking a background medication, which background medication is an anti-fibrotic agent, a long-acting muscarinic antagonist (LAMA), a long-acting beta-agonist (LABA) or an inhaled corticosteroid (ICS). The patient may not be taking a background medication which is a muscarinic antagonist or a beta-agonist.

The compound is ensifentrine or a pharmaceutically acceptable salt thereof. Pharmaceutically acceptable salts are well known to the skilled person. Typically, the compound is ensifentrine (i.e. ensifentrine free base).

The method comprises administering the compound to the patient by inhalation. A pharmaceutical composition comprising the compound and one or more pharmaceutically acceptable excipients or diluents is typically administered to the patient by inhalation, for instance by nebuliser, pressurised metered dose inhaler (pMDI) or dry powder inhaler (DPI).

Preferably, the method comprises administering the compound to the patient by inhalation from a nebuliser. Nebulisers aerosolise a liquid pharmaceutical composition into an aerosol that is inhaled into a patient's respiratory tract. Examples of nebulisers include a soft mist nebuliser, a vibrating mesh nebuliser, a jet nebuliser and an ultrasonic wave nebuliser. Suitable nebuliser devices include the Philips I-Neb™ (Philips), the Philips SideStream (Philips), the AeroNeb® (Philips), the Philips InnoSpire Go (Philips), the Pari LC Sprint (Pari GmbH), the AERxR™ Pulmonary Delivery System (Aradigm Corp) and the Pari LC Plus Reusable Nebuliser (Pari GmbH). The nebulizer may for instance be a PARI LC Sprint jet nebulizer with a PARI Vios® PRO Aerosol Delivery System PARI BOY® compressor. The compound may be inhaled via the nebuliser for from 1 to 15 minutes.

Typically, the method comprises administering the compound to the patient once, twice or three times per day, for instance twice or three times per day. The compound may be administered to the patient by inhalation once, twice or three times a day. Preferably the method comprises administering the compound to the patient by inhalation twice a day. The method may comprise administering a first dose of the compound in the morning (for instance within 3 hours following waking) and a second dose of the compound in the evening (for instance within 3 hours before bed). Typically, the morning and evening doses are administered from 10 to 14 hours apart, for instance about 12 hours apart.

The compound may be used in any suitable therapeutically effective amount. Typically, the daily dose of the compound is from 0.1 to 40 mg or from 0.1 to 20 mg. Typically, the method comprises administering a total daily dose of the compound of from 0.5 to 10 mg. Preferably, the total daily dose of the compound (e.g. ensifentrine free base) is from 5 to 7 mg, for instance about 6 mg per day. As used herein, the term “about” may represent a variation of ±10% of the stated value. The total daily dose of the compound may be 6.0 mg.

Typically, the compound is administered twice a day in two separate doses which are the same or similar. The method may comprise administering the compound to the patient twice a day in a first dose of from 1 to 10 mg and a second dose of from 1 to 10 mg. For instance, the method may comprise administering the compound to the patient twice a day in a first dose of from 1 to 5 mg and a second dose of from 1 to 5 mg. Typically, the method may comprise administering the compound to the patient twice a day in a first dose of from 2 to 4 mg and a second dose of from 2 to 4 mg.

The method may comprise administering two doses of about 2 mg, about 3 mg, about 4 mg, about 5 mg or about 6 mg ensifentrine free base to the patient per day by inhalation. Preferably, the method comprises administering two doses of about 3 mg ensifentrine free base to the patient per day by inhalation. The method preferably comprises administering a dose of about 3 mg of the compound to the patient twice a day (3 mg BID) by inhalation. More preferably, the method comprises administering by nebuliser a dose of about 3 mg the compound to the patient twice a day. Each dose may be 3.0 mg free base ensifentrine administered by nebulizer.

Typically, the method comprises administering the compound to the patient at least once per day for at least 8 weeks. The compound may be administered to the patient at least once per day for at least 16 weeks, preferably for at least 24 weeks. The compound may be administered daily to the patient for at least 1 year. The method may comprise administering the compound to the patient at least once every 24 hours, preferably at least twice every 24 hours, for at least 8 weeks, preferably for at least 16 weeks, more preferably for at least 24 weeks. The compound is typically used as a maintenance therapy.

The compound is preferably administered as a suspension formulation, i.e. a suspension of particles comprising the compound in a diluent. The compound may alternatively be delivered as a dry powder, for instance a dry powder comprising particles comprising the compound and particles of a carrier such as lactose.

The method typically comprises administering an inhalable pharmaceutical composition comprising a suspension of particles of the compound in a diluent. The particles comprising the compound typically have a particle size distribution with a Dv50 of from 0.5 μm to 5.0 μm. The particles preferably have a Dv50 of from 1.0 μm to 2.0 μm.

Particle sizes are described herein by reference to the Dv50 value, which is the median particle size for a volume distribution. Thus, half the volume of the particles have diameters of less than the Dv50 value and half the volume of the particles have diameters of greater than the Dv50 value. This is a well-known manner in which to describe particle size distributions.

The technique used to measure the Dv50 values as stated herein is typically laser diffraction. The particle size distribution of the particles comprising the compound may be as measured by laser diffraction using a wet powder dispersion system. For instance, the particle size distribution can be measured by laser diffraction using a Malvern Spraytec in conjunction with a wet dispersion cell. Typically, the instrument parameters for the Malvern Spraytec are as follows:
particle—standard opaque particle;
refractive index Particle—1.50;
refractive index (imaginary)—0.50;
density of particle—1.00;
refractive index of dispersant—1.33;
controller unit—1000 RPM;
measurement type—timed;
initial sampling time—30 s;
obscuration—20%-30%;
dispersant—1% Polysorbate 20 in deionised water.

The particles comprising the compound typically comprise ensifentrine (i.e. ensifentrine free base). The particles may comprise at least 90 wt % ensifentrine free base relative to the total weight of the particles. The particles may comprise at least 99 wt % ensifentrine. The particles may consist of ensifentrine.

The concentration of particles comprising the compound in the inhalable pharmaceutical composition is typically from 0.1 to 5.0 mg/mL, preferably from 0.1 to 2.5 mg/mL, more preferably from 1.0 to 2.0 mg/mL.

The inhalable pharmaceutical composition typically further comprises one or more tonicity adjusters, one or more buffers and one or more surfactants. The tonicity adjuster is typically sodium chloride.

Examples of buffers include a citrate buffer, a phosphate buffer, an acetate buffer, and a bicarbonate buffer. Preferably, the buffer is a phosphate buffer, for instance sodium dihydrogen phosphate dihydrate and/or disodium phosphate dihydrate.

Examples of surfactants include lecithin, oleic acid, polyoxyethylene glycol alkyl ethers (for instance PEG 300, PEG 600, PEG 1000, Brij 30, Brij 35, Brij 56, Brij 76 and Brij 97), polypropylene glycol (for instance PPG 2000), glucoside alkyl ethers, polyoxyethylene glycol octylphenol ethers, polyoxyethylene glycol alkylphenol ethers, glycerol alkyl esters, polyoxyethylene glycol sorbitan alkyl esters (polysorbates, for instance polysorbate 20, polysorbate 40, polysorbate 60 and polysorbate 80), sorbitan alkyl esters (for instance sorbitan monolaurate (Span 20), sorbitan monooleate (Span 80) and sorbitan trioleate (Span 85)), cocamide MEA, cocamide DEA, dodecyldimethylamine oxide, block copolymers of polyethylene glycol and polypropylene glycol (poloxamers), block copolymers of polyethylene glycol and polypropylene oxide (for instance Pluronic surfactants), polyvinyl pyrrolidone K25, polyvinyl alcohol, oligolactic acid, sodium dioctyl sulfosuccinate and polyethoxylated tallow amine (POEA).

Preferably, the one or more surfactants comprise a polysorbate and/or a sorbitan alkyl ester. The one or more surfactants may for instance comprise polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate) or polysorbate 80 (polyoxyethylene (20) sorbitan monooleate). The one or more surfactants may for instance comprise sorbitan monolaurate (Span 20), sorbitan monooleate (Span 80) or sorbitan trioleate (Span 85). Preferably, the sterile liquid vehicle comprises polysorbate 20 and/or sorbitan monolaurate (Span 20).

For instance, the method may comprise administering to the patient an inhalable liquid pharmaceutical composition comprising:
water;
particles consisting of ensifentrine free base at a concentration of from 0.1 to 20 mg/mL;
one or more tonicity adjusters at a total concentration of from 1.0 to 15 mg/mL;
one or more buffers at a total concentration of from 0.1 to 4 mg/mL; and
one or more surfactants at a total concentration of from 0.05 to 3 mg/mL.

The inhalable liquid pharmaceutical composition may comprise:
water;
particles consisting of ensifentrine free base at a concentration of from 0.5 to 6 mg/mL;
sodium chloride at a concentration of from 5 to 12 mg/mL;
sodium dihydrogen phosphate dihydrate at a concentration of from 0.3 to 2 mg/mL;
disodium phosphate dihydrate at a concentration of from 0.3 to 2 mg/mL;
polysorbate 20 at a concentration of from 0.1 to 1.5 mg/mL; and
sorbitan monolaurate at a concentration of from 0.01 to 0.5 mg/mL.

A side effect of some anti-fibrotic treatments is gastrointestinal adverse events such as diarrhoea. These side effects can prevent patients from taking the compound, and may reduce patient compliance. This is particularly the case for patients already suffering from, or susceptible to, gastrointestinal disorders such as diarrhoea.

Inhaled ensifentrine has been found not to be associated with diarrhoea in patients. Accordingly, the compound may be used to treat pulmonary fibrosis in a patient suffering from, or susceptible to a gastrointestinal disorder. The patient may be susceptible to, or suffering from, diarrhoea. The patient may be suffering from ulcerative colitis, Crohn's disease, microscopic colitis, celiac disease, irritable bowel syndrome, bile acid malabsorption, or diverticulitis.

The compound may be used in combination with a second active agent. The compound may be administered separately or simultaneously with the second active agent. The patient may already be taking a second active agent as a background therapy for pulmonary fibrosis. Alternatively, treatment with the second active agent may start at around the same time as treatment with the compound. The compound and the second active agent may be administered in a fixed combination.

The second active agent is typically an anti-fibrotic agent, a muscarinic receptor antagonist, a beta-adrenergic receptor agonist or an inhaled corticosteroid. The compound may accordingly be used in combination with an anti-fibrotic agent, a muscarinic receptor antagonist, a beta-adrenergic receptor agonist or an inhaled corticosteroid.

Typically, the second active agent is anti-fibrotic agent. The anti-fibrotic agent may be pirfenidone, nintedanib or a pharmaceutically acceptable salt thereof. Preferably, the anti-fibrotic agent is pirfenidone.

The second active agent may be administered by inhalation or orally. Typically, the anti-fibrotic agent is administered orally. For instance, the patient may be taking an oral anti-fibrotic agent such as pirfenidone as a background medication.

The second active agent may be a long-acting muscarinic receptor antagonist (LAMA), a long-acting beta-adrenergic receptor agonist (LABA) or an inhaled corticosteroid. Examples of LAMAs include aclidinium, darotropium, tiotropium, glycopyrrolate, umeclidinium and pharmaceutically acceptable salts thereof. Examples of LABAs include salmeterol, formoterol, indacaterol, vilanterol, olodaterol, abediterol, carmoterol and pharmaceutically acceptable salts thereof. Examples of inhaled corticosteroids include beclomethosone, budesonide, fluticasone propionate, ciclesonide, mometasone, fluticasone furoate and pharmaceutically acceptable salts thereof.

The compound may alternatively be used as a monotherapy. For instance, the compound may be for use in treating pulmonary fibrosis (e.g. idiopathic pulmonary fibrosis) as the sole active agent. In some embodiments, the compound is not administered in combination with a muscarinic receptor antagonist or a beta-adrenergic receptor agonist.

It is a finding of the invention that ensifentrine may be used in combination with an anti-fibrotic agent. The invention accordingly also provides a composition comprising (i) a compound which is ensifentrine or a pharmaceutically acceptable salt and (ii) an anti-fibrotic agent.

The composition is typically a pharmaceutical composition. The composition may further comprise one or more pharmaceutically acceptable carriers, diluents or excipients. The one or more pharmaceutically acceptable carriers, diluents or excipients may be as defined herein. The composition may be administered as described herein. For instance, the composition may be administered by inhalation.

The two active ingredients may be in fixed or free combination. Typically, the two active ingredients are in fixed combination. For instance, they may be intermixed. The two active ingredients may be present in an inhalable liquid pharmaceutical composition.

Typically, the anti-fibrotic agent is pirfenidone, nintedanib or a pharmaceutically acceptable salt thereof. The anti-fibrotic agent is preferably pirfenidone.

The composition may be for use in a method for treating or preventing pulmonary fibrosis as defined herein.

The invention also provides an anti-fibrotic agent (for instance pirfenidone) for use in a method of treating pulmonary fibrosis in a patient in combination with a compound which is ensifentrine or a pharmaceutically acceptable salt thereof. The method may be as further defined herein.

The invention also provides a method of preventing or treating pulmonary fibrosis in a patient by improving lung function, or by preventing or reducing a reduction in lung function, which method comprises administering by inhalation a therapeutically effective amount of a compound which is ensifentrine or a pharmaceutically acceptable salt thereof to the patient.

Further provided by the invention is use of a compound which is ensifentrine or a pharmaceutically acceptable salt thereof in the manufacture of a medicament for a method of treating pulmonary fibrosis by improving lung function, or by preventing or reducing a reduction in lung function, wherein the method comprises administering the compound to the patient by inhalation.

The invention also provides a method of treating IPF in a patient, which method comprises administering by inhalation a therapeutically effective amount of a compound which is ensifentrine or a pharmaceutically acceptable salt thereof to the patient.

Further provided by the invention is use of a compound which is ensifentrine or a pharmaceutically acceptable salt thereof in the manufacture of a medicament for a method of treating IPF, wherein the method comprises administering the compound to the patient by inhalation.

The invention is described in more detail by the following Examples.

### EXAMPLES

### Example 1—Effect of Ensifentrine on Inflammatory Mediators

### Study Design

A clinical study was conducted to determine the efficacy of ensifentrine in treating COPD compared with placebo. Ensifentrine was administered by nebuliser at a dose of 3 mg twice daily (BID) for 24 weeks. The study was a multi-centre, randomized, double-blind, parallel-group, placebo-controlled trial with around 800 patients and 5:3 randomization.

The study population included patients aged 40-80 years with moderate to severe COPD (FEV₁ 30%-70% p.n., FEV₁/forced vital capacity (FVC) ratio <0.7, with mMRC ≥2). The randomization stratified (a) the use of stable background maintenance LAMA or LABA therapy use (approx. 50%. yes or no) and (b) cigarette smoking (current or former). Inhaled corticosteroid (ICS) maintenance therapy was permitted in up to 20% of patients under certain provisions.

The effect of ensifentrine on the change in blood IL-6 and IL-8 was measured.

### Formulation

The investigational product and placebo were provided in 2.5 mL unit dose format in an ampule and administered via a nebuliser. The formulation of the investigational product (ensifentrine suspension formulation) and placebo are shown in Table 1 below.

TABLE 1

| Constituent | Concentration (mg/mL) |
|---|---|
| Ensifentrine particles (RPL554) | 1.2 (for the active) |
| | or 0 (for Placebo) |
| Polysorbate 20 (Tween 20) | 0.50 |
| Sorbitan Monolaurate (Span 20) | 0.05 |
| Sodium Dihydrogen Phosphate Dihydrate | 0.744 |
| Disodium Hydrogen Phosphate Dihydrate | 0.853 |
| Sodium Chloride | 8.60 |
| Water | q.s. to 1 mL |

### Results

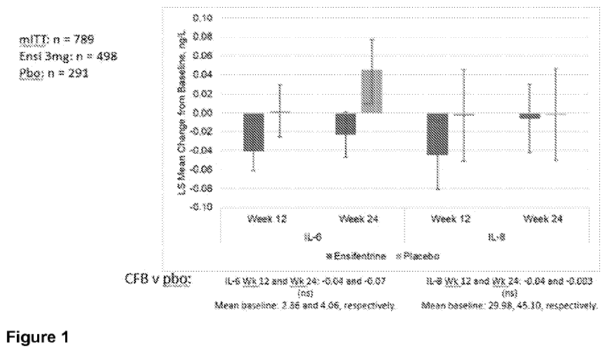

The effect of inhaled ensifentrine on the inflammatory biomarkers interleukin 6 (IL-6) and IL-8 compared with placebo is shown in FIG. 1. Inhaled ensifentrine was found to reduce blood IL-6 and IL-8 in patients.

### Conclusion

It has been found that inhaled ensifentrine 3 mg twice daily (BID) caused reduction in blood IL-6 and IL-8 in patients, consistent with the profile of an anti-fibrotic drug.

### Example 2—Effect of Ensifentrine in a Rodent Model of IPF

The anti-fibrotic activity of ensifentrine is assessed using a rodent model of bleomycin-induced lung fibrosis.

Bleomycin is administered to anaesthetised rodents to induce lung fibrosis. Ensifentrine is then administered to the test rodents by inhalation for a period, and the rodents are subsequently killed. Lung samples are obtained for histological studies to determine fibrotic area.

Ensifentrine is determined to have an anti-fibrotic effect in bleomycin-induced lung fibrosis.

### Example 3—Effect of Ensifentrine on Bleomycin-Induced Pulmonary Fibrosis

### Study Overview

An in vivo study was conducted to determine the efficacy of ensifentrine in the treatment of bleomycin-induced pulmonary fibrosis in rats in comparison to a control. Rats were administered bleomycin to induce pulmonary fibrosis. After a week, they then received a daily inhaled dosage of ensifentrine or a control over a period of two weeks. At the end of the two weeks, the rats were sacrificed and their airways were analysed for Bronchoalveolar lavage (BAL) differential cell counts and TGF-β concentration.

The BAL differential cell count provided a total inflammatory cell count so that changes in inflammation following treatment could be observed. TGF-β is a key cytokine in the development of tissue fibrosis. Macrophages and other immune cells produce TGF-β, which induces the differentiation of fibroblasts to myofibroblasts and increases TGF-β expression. Overexpression of active TGF-β1 in the lungs of rodents causes severe and progressive tissue fibrosis, including fibroblastic foci and honeycomb formation. Inhibiting TGF-β1 may be central to the anti-fibrotic effect of a drug.

### Study Design

At day 0, the rats were subjected to an intratracheal challenge of either 0.9% w/v saline or bleomycin, the latter used to induce pulmonary fibrosis.

Starting on day 7, two groups of 12 rats from the bleomycin challenge group were administered an inhaled dose of ensifentrine at either 3 mg/kg (Experiment 1) or 10 mg/kg (Experiment 2) on a daily basis for two weeks, terminating on day 21. The ensifentrine was administered as a suspension in the vehicle suspension article.

Two control experiments were also conducted. Control 1 subjected 12 rats from the saline challenge group to daily administration of the vehicle suspension article via inhalation for the full 21 days. Control 2 subjected 12 rats from the bleomycin challenge group to daily administration of the vehicle suspension article via inhalation for the full 21 days. The experimental set-up is summarised in Table 2 below.

TABLE 2

| | Intratracheal | | Targeted | |
|---|---|---|---|---|
| | Challenge | | Inhaled Dose | Treatment |
| Experiment | (Day 0) | Treatment | (mg/kg/day) | Frequency |
| Experiment 1 | Bleomycin | Ensifentrine | 3 | QD from |
| | | | | D 7 |
| Experiment 2 | Bleomycin | Ensifentrine | 10 | QD from |
| | | | | D 7 |
| Control 1 | Saline (0.9% | Placebo | — | QD from |
| | w/v) | suspension | | D 0 |
| | | article | | |
| Control 2 | Bleomycin | Placebo | — | QD from |
| | | suspension | | D 0 |
| | | article | | |

At the end of the 21-day period, the rats were sacrificed and the BAL differential cell counts and the TGF-β concentration data were collected.

### Results

The BAL differential cell counts at day 21 showed a reduction in total inflammatory cell and macrophage infiltration into the rat airways following treatment with ensifentrine at 3 mg/kg/day (Experiment 1) and 10 mg/kg/day (Experiment 2), in comparison to the bleomycin Control 2.

The TGF-β concentration at day 21 was also lower in the rats following ensifentrine treatment compared to those of the bleomycin Control 2.

### Conclusion

It has been found that inhaled ensifentrine at 3 mg/kg and 10 mg/kg caused a reduction in BAL differential cell count and TGF-β concentration in rats with bleomycin-induced pulmonary fibrosis. It has therefore been found that ensifentrine administered via inhalation is effective in the treatment of bleomycin-induced pulmonary fibrosis.

### Example 4—Comparison of Ensifentrine and Nintedanib

### Study Design

An in vivo study was conducted to compare the efficacy of ensifentrine and nintedanib in the treatment of bleomycin-induced pulmonary fibrosis in rats. Following the same initial experimental procedure as in Example 3, 12 rats from the bleomycin challenge group were administered a daily dose of 100 mg/kg of nintedanib via an oral gavage for two weeks, starting from day 7 (Experiment 3). A new control experiment was also conducted (Control 3), administering the vehicle suspension article orally in an aqueous solution. This experimental design is summarised in Table 3 below.

At day 21, the rats were sacrificed and their airways were analysed to calculate the TGF-β concentration. The results were compared to those of Experiments 1 and 2 (Example 3).

TABLE 3

| | Intratracheal | | Targeted | |
|---|---|---|---|---|
| | Challenge | | Inhaled Dose | Treatment |
| Experiment | (Day 0) | Treatment | (mg/kg/day) | Frequency |
| Experiment 3 | Bleomycin | Nintedanib | 100 | QD from |
| | | | | D 7 |
| Control 3 | Bleomycin | Placebo | — | QD from |
| | | suspension | | D 0 |
| | | article | | |

### Results

The TGF-β concentration in the rat lungs following treatment with ensifentrine (Experiments 1 and 2) was lower than the TGF-β concentration following treatment with nintedanib (Experiment 3).

### Conclusion

It has been found that inhaled ensifentrine at 3 mg/kg/day or 10 mg/kg/day causes a greater reduction in TGF-β concentration than orally administered nintedanib at 100 mg/kg/day in rats with bleomycin-induced pulmonary fibrosis.

1. A compound for use in a method of treating idiopathic pulmonary fibrosis in a patient, which compound is ensifentrine or a pharmaceutically acceptable salt thereof,
wherein the method comprises administering the compound to the patient by inhalation.

2. A compound for use in a method of treating pulmonary fibrosis in a patient by improving lung function, or by preventing or reducing a reduction in lung function, which compound is ensifentrine or a pharmaceutically acceptable salt thereof,
wherein the method comprises administering the compound to the patient by inhalation.

3. A compound for use according to claim 2, wherein the pulmonary fibrosis is idiopathic pulmonary fibrosis.

4. A compound for use according to any one of the preceding claims, wherein the compound improves lung function in the patient, preferably wherein the compound increases the forced vital capacity of the patient.

5. A compound for use according to any one of claims 1 to 3, wherein the compound prevents a reduction of lung function in the patient, preferably wherein the compound prevents or reduces a decline in forced vital capacity of the patient.

6. A compound for use according to any one of the preceding claims, wherein the compound treats the pulmonary fibrosis by preventing or reducing fibrosis, preferably wherein the compound treats the pulmonary fibrosis by reducing fibrosis.

7. A compound for use according to any one of the preceding claims, wherein the compound decreases the frequency and/or severity of pulmonary fibrosis exacerbations in the patient suffering from pulmonary fibrosis.

8. A compound for use according to any one of the preceding claims, wherein the patient is suffering from idiopathic pulmonary fibrosis (IPF) and the compound decreases the frequency and/or severity of acute exacerbation of IPF (AE-IPF) in the patient.

9. A compound for use according to any one of the preceding claims, wherein the method comprises administering the compound to the patient by inhalation from a nebuliser.

10. A compound for use according to any one of the preceding claims, wherein the compound is ensifentrine.

11. A compound for use according to any one of the preceding claims, wherein the method comprises administering the compound to the patient once, twice or three times per day.

12. A compound for use according to any one of the preceding claims, wherein the method comprises administering the compound to the patient twice a day.

13. A compound for use according to any one of the preceding claims, wherein the method comprises administering to the patient a total daily dose of the compound of from 0.1 to 20 mg, preferably from 0.5 to 10 mg.

14. A compound for use according to any one of the preceding claims, wherein the method comprises administering the compound to the patient twice a day in a first dose of from 2 to 4 mg and a second dose of from 2 to 4 mg.

15. A compound for use according to any one of the preceding claims, wherein the method comprises administering a dose of about 3 mg the compound to the patient twice a day (3 mg BID), preferably wherein the method comprises administering by nebuliser a dose of about 3 mg the compound to the patient twice a day.

16. A compound for use according to any one of the preceding claims, wherein the method comprises administering the compound to the patient at least once per day for at least 8 weeks, preferably for at least 16 weeks, more preferably for at least 24 weeks.

17. A compound for use according to any one of the preceding claims, wherein the patient is susceptible to, or suffering from, diarrhoea.

18. A compound for use according to claim 17, wherein the patient is suffering from ulcerative colitis, Crohn's disease, microscopic colitis, celiac disease, irritable bowel syndrome, bile acid malabsorption, or diverticulitis.

19. A compound for use according to any one of the preceding claims, wherein the method comprising administering an inhalable pharmaceutical composition comprising a suspension of particles of the compound in a diluent.

20. A compound for use according to any one of the preceding claims, wherein the compound is used in combination with a second active agent, which second active agent is an anti-fibrotic agent, a muscarinic receptor antagonist, a beta-adrenergic receptor agonist or an inhaled corticosteroid.

21. A compound for use according to claim 20, wherein the second active agent is an anti-fibrotic agent.

22. A compound for use according to claim 21, wherein the anti-fibrotic agent is pirfenidone, nintedanib or a pharmaceutically acceptable salt thereof.

23. A compound for use according to claim 21 or claim 22, wherein the anti-fibrotic agent is administered orally.

24. A composition comprising (i) a compound which is ensifentrine or a pharmaceutically acceptable salt thereof and (ii) an anti-fibrotic agent.

25. A composition according to claim 24, wherein the anti-fibrotic agent is pirfenidone, nintedanib or a pharmaceutically acceptable salt thereof.

26. A composition as defined in claim 24 or 25 for use in a method for treating pulmonary fibrosis as defined in any one of claims 1 to 22.

27. An anti-fibrotic agent as defined in claim 24 or 25 for use in a method of treating pulmonary fibrosis in a patient in combination with a compound which is ensifentrine or a pharmaceutically acceptable salt thereof.

28. A method of treating idiopathic pulmonary fibrosis in a patient, which method comprises administering by inhalation a therapeutically effective amount of a compound which is ensifentrine or a pharmaceutically acceptable salt thereof to the patient.

29. A method of treating pulmonary fibrosis in a patient by improving lung function, or by preventing or reducing a reduction in lung function, which method comprises administering by inhalation a therapeutically effective amount of a compound which is ensifentrine or a pharmaceutically acceptable salt thereof to the patient.

30. Use of a compound which is ensifentrine or a pharmaceutically acceptable salt thereof in the manufacture of a medicament for a method of treating idiopathic pulmonary fibrosis in a patient, wherein the method comprises administering the compound to the patient by inhalation.

31. Use of a compound which is ensifentrine or a pharmaceutically acceptable salt thereof in the manufacture of a medicament for a method of treating pulmonary fibrosis in a patient by improving lung function, or by preventing or reducing a reduction in lung function, wherein the method comprises administering the compound to the patient by inhalation.
